(19) United States
(12) Patent Application Publication
(10) Pub. No.: US 2023/0064841 A1
(43) Pub. Date: Mar. 2, 2023

(54) MOBILE INTEGRATED PATIENT SUPPORT APPARATUS

(71) Applicant: Ahmed Abdelsalam Mohamed ABOHASHEM, Madinah Monawarah (SA)

(72) Inventors: Ahmed Abdelsalam Mohamed ABOHASHEM, Madinah Monawarah (SA); Moaz Abdelsalam Mohamed ABOHASHEM, Sharqia (EG)

(21) Appl. No.: 17/794,316

(22) PCT Filed: Jul. 21, 2020
(86) PCT No.: PCT/IB2020/056855

§ 371 Date: Jul. 21, 2022

(51) Int. Cl. A61G 7/057; A61G 12/00; G16H 40/63; A61G 7/05

(52) U.S. Cl. CPC A61G 7/057; A61G 12/008; G16H 40/63; A61G 7/0524

(57) ABSTRACT

A patient support apparatus, mobile integrated critical care unit. a patient bed includes more than a system such as a ventilation system, monitoring system, and a bar as a holder for syringe and infusion pumps implemented with their power supply and supported with examination light. Left head side rail includes monitoring system interface and the rear side of the same side rail includes the docking area for patient probes which in communicate with monitoring electronic system fixed inside the side rail. Ventilation system interface at the right head side rail. Inspiration and expiration modules coupled to the backside of the patient backrest board connected to the patient through a breathing circuit. Ventilation system control box, ventilation power box, gases control box, located at the unit base frame. A wall-mounted monitor displays systems data. The unit provided with blower and back up oxygen cylinder to support the ventilation system.

### REFERENCES CITED

### U.S. Patent Documents

| | | |
|---|---|---|
| U.S. Pat. No. 4,715,385 | December 1987 | Cudahy et al. |
| U.S. Pat. No. 5,339,805 | August 1994 | Carter |
| U.S. Pat. No. 5,390,666 | February 1995 | Kimm et al. |
| U.S. Pat. No. 6,330,926 | December 2001 | Heimbrock et al. |
| U.S. Pat. No. 7,090,014 B2 | August 2006 | Vogel et al. |
| US 2008/0141459 A1 | June 2008 | Hamberg et al. |
| U.S. Pat. No. 7,492,111 B2 | February 2009 | Soukup |
| US 2009/0188731 A1 | July 2009 | Zerhusen et al. |
| US 2009/0313758 A1 | December 2009 | Menkedick et al. |
| U.S. Pat. No. 7,647,659 B2 | January 2010 | Frolik et al. |
| US 2010/0078026 A1 | April 2010 | Andrieux et al. |
| US 2011/0015842 A1 | January 2011 | Kume et al. |
| US 2011/0138323 A1 | June 2011 | Skidmore et al. |
| US 2011/0209707 A1 | September 2011 | Terhark |
| US 2011/0213215 A1 | September 2011 | Doyle et al. |
| U.S. Pat. No. 8,112,836 A1 | February 2012 | Tesar et al. |
| U.S. Pat. No. 9,707,143 B2 | March 2013 | Thodupunuri et al. |
| U.S. Pat. No. 8,452,508 B2 | March 2013 | Frolik et al. |
| U.S. Pat. No. 8,439,032 B2 | May 2013 | Andrieux et al. |
| U.S. Pat. No. 8,959,680 B2 | February 2015 | Tesar et al. |
| U.S. Pat. No. 10,016,325 B2 | April 2017 | Ribble et al. |
| U.S. Pat. No. 9,707,369 B2 | July 2017 | Desilva et al. |
| U.S. Pat. No. 10,076,458 B2 | September 2018 | McClellan et al. |
| U.S. Pat. No. 10,096,137 B2 | October 2018 | Rajagoplana et al. |
| | | -20- |
| U.S. Pat. No. 10,206,835 B2 | February 2019 | Jurka |

### Other Publications

Bene Vision N1 patient monitor service manual, Shenzhen Mindray Bio-Medical Electronics CO., Ltd P/N: 046-011649-00 (3.0).
Bergman, Petterson, Chaboyer, Carlstrõm & Ringdal. Safety hazards during intrahospital transports a prospective observational study Crit Care Med 2017 DOI: 10.1097/CCM.0000000000002653
Ringdal, Chaboyer, Warr\acute{e}n Stomberg Intrahospital transports of critically ill patients: critical care nurses' perceptions. Nurs Crit Care 2015
Aust H, Kranke P, Eberhart L H, Afshari A, Weber F, Brieskorn M, Heine J, Arndt C, Rüsch D. Impact of medical training and clinical experience on the assessment of oxygenation and hypoxaemia after general anaesthesia: an observational study. J Clin Monit Comput. 2015 June; 29(3):415-26.
Evidence-based design in an intensive care unit: End-user perceptions Mauricio Ferri, David A Zygun, Alexandra Harrison and Henry T Stelfox Ferri et al. BMC Anesthesiology (2015) 15:57
Transport of critically ill patient Dr. Masthanamma. C Dr. Raju. P.S.N.

### FIELD OF THE INVENTION

In general the present invention relates to the field of patient support apparatuses Such as hospital bed, patient monitoring system for providing continuous monitoring of patient vital signs, mechanical ventilation system assisting patient breathing. In particular, the invention relates to critical care patient support apparatuses.

### BACKGROUND

Present critical care staff and patient suffering a lot of hazards and disadvantages inside patient's room and during patient procedures, for example, Intrahospital Transport (IHT), this frequent complex and sensitive procedures requires more features to provide more high quality and safety standers to patient and staff, also it's a time-consuming task for staff working in critical care department, typically where nurses have to execute various tasks with high physical and psychological requirements.

A study published in nursing critical care indicates that increased workload in an intensive critical care unit (ICU) often keeps nurses from taking necessary work breaks leading to a high level of stress, intra hospital transport often contributes to these higher stress level for staff, creating operational inefficiencies. Seeking to provide easier, faster, and safer (IHT) is a major concern in this disclosure.

A questionnaire study to critical care nurses in two Swedish ICUs (86 critical care nurse who answered), there comments about (IHT): 22% it makes demand and time consuming, 20% it's a moment of stress, 19% it's a burden of work for staff remaining in the ICU, 14% taking care of other patient comes to a halt as well and 5% its well-functioning.

Another study in critical care medicine titled (safety hazards during intrahospital transport), the study clarifies that hazards during (IHT) comprise hazards related to the patient, the sicker patient, the greater chance of problem and hazards related to the system which comprises (hazards related to staff and organization—hazards related to equipment and tools). In present disclosure aiming to minimize hazards related to equipment leading to decrease other hazards related to the system. In the same study during 51 (IHT) found 365 hazards, the biggest part of those hazards comes from equipment and technologies 124 hazards, the study suggested (Time Out) solution for safer (IHT). (Time Out) is a procedure followed in operation theater before they start, presenting themselves and discuss the steps of procedures. (Time Out) is a hospital staff solution, the present disclosure may be considered as one of the technology solutions aiming to improve the safety of (IHT), making it more faster and comfortable not only for the patient but for staff also by reducing the stress on them and producing enhancement of productiveness.

Another study in the same field suggests some key things to be considered during (IHT), one of these key things Technology and equipment, should be standardized, mobile, acceptance by staff, adapted to workflow, stability in the functioning and physical robustness. Another key thing is innovation, the study suggests during (IHT) if we look to the future in (IHT) one connected device during all steps and procedures of (IHT), it's important that the ergonomic situation with monitoring and ventilation is acceptable and it should be integrated with the whole system in the environment.

During (IHT), the use of more equipment could result in a higher probability of equipment related problems that might divert the attention of the staff from patient to device. Currently, nurses and other hospital staff hang pumps and/or other equipment and accessories on the top edge of the foot, headboards, or side rails, since these edges were not designed to support the hanging of the patient equipment. This current practice reduce access to control keypads and cause damage edges of the foot, headboards and side rails, the hanging equipment may fall from edges and damage too, also such a practice divert staff attention to observe both patient and hanging equipment leading to decrease patient safety and increasing hazards during procedures.

Besides, the ICU room of prior art relies on a dual pendent system, it's occupying a large space around the patient, leading to limits staff and bed movements, some positions of the bed for particular procedures couldn't be done due to the limited area between the dual pendent system. A study titled (evidence-based design in an intensive care unit end-user perception), a participant commented on equipment usability seventeen times in early phase, providers identified negative aspects of new equipment usability such as problems getting used to the dual pendent mounted system, and the comment is (the arms are very frustrating because before you could decide where a ventilator was, where your IV pumps and to the foot of the bed so it would be out of everyone's way).

One of this study messages is (large space required attention to the tradeoffs of size, large space facilitate family presence and increase the perception of high quality of care, however, safety concern about increased distance between providers and patients require careful consideration). In the prior art of the ICU room a large space occupied with equipment around the patient giving the staff a small space to deal with patients leading to more stress to the staff and wasting more of their time because they can't move freely around the patient.

One of the problems facing ICU staff is having a web of cables, wires, and hoses between the patient and other equipment on bed's both sides, annoying the staff during most procedures such as patient examination, cleaning, X-Ray procedure by the portable machine and during preparing the patient for Intrahospital Transport, causing more stress on the staff. Besides the time delays which may be encountered when adding sensors to the monitoring system, in the prior art also leave much to be desired with respect to cable management, a large number of cables extended between the patient and the monitor, in the past, there has been at least one cable added for each parameter monitored, this array of cables and hoses interfaces with the movement of staff around the patient's bed. The greater number of cables, the greater the risk that someone will accidentally disrupt one of them.

Two major considerations in the design of the monitoring system have been ease and speed of the system, it's particularly undesirable to connect sensors to the patient or disconnect them immediately before transportation or administration of critical procedures.

### SUMMARY

After clarifying some of the disadvantages and hazards which staff and patient facing in ICU department, the present invention is a critical care patient apparatus comprise more than a system has one or more of the following features, which alone or in any combination may comprise a patentable subject matter. critical care room contains four main systems: patient monitoring system, patient mechanical ventilation system, syringe, and infusion pumps (injection system) and patient bed, the present disclosure is about to integrate all these systems to patient bed components, in a way support the staff, patient and hospital system more than before. In the present embodiment, the new ICU room may contain only our disclosure unit plus a wall-mounted monitor.

An object of the present invention provides a patient support apparatus, in one embodiment of the disclosure, which may be not only a bed or a stretcher but also could be used as a mobile integrated critical care unit, as a result of providing most of critical care patient's needs inside patient room.

The directions of unit side rails and boards are determined from a patient perspective view.

Patient monitoring system located at the left head side rail of the bed (unit) comprises, a connection port at docking area, patient probes implemented with sensors, monitoring electronic system fixed inside the side rail, and monitoring system interface (display). The electronic system communicates with the patient to receive the data via patient probes and communicate with the system display which configured to monitor the patient vital signs data.

The patient vital signs data are determined or received by an electronic system configured to process and present related information to the user via two interfaces, first one, is monitoring system display at left head side rail. Second, the interface is the wall-mounted monitor which may include a processor, memory, and storage, configured to receive and monitor the data from the electronic system. The monitoring system implemented with a software program, hardware circuitry (electronic system), pumps, valves, sensors, and a combination thereof.

In another aspect of the invention, the right head side rail includes the ventilation system display which may display patient parameters and ventilation parameters, sensors reading, ventilation system performance, and/or calculated parameters. Both head side rails contain a control keypad to enable the user from operating the unit to any needed position. The unit base frame may include a ventilation control box, gases control box with blower, holders for a backup oxygen cylinder, unit lifting actuators, drive wheel system, power boxes, batteries, and other components. Patient breathing circuit (patient limb) connect inspiration and expiration modules to the patient, the modules located at the backside of the patient backrest board, so to be nearby patient's head and chest during all unit positions and movements without any barriers.

The head-end section of the unit may be provided with a removable bar to a holder for syringe and infusion pumps and the lifting handle. The bar may be provided with DC power connections for supplying syringe and infusion pumps. The unit may include an additional piece of patient care equipment such as an examination light located at the end of the bar attached with a flexible arm. The unit supported with a drive wheel system configured to enable the user to transport the unit manually and/or with drive wheel system such as (the Intell® I drive transport system sold by Hill-Rom Company) or (I Drive Power® transport system sold by Linet.s.pol Company).

The unit supplied with air blower and removable backup oxygen cylinder to support mechanical ventilation during patient transport and/or any emergency event could happen to the main gases supply, also the unit supported with a long-life battery to run all the unit functions during (IHT), other procedures and/or any emergency event could happen to the main power supply.

A wall-mounted monitor (interface) to store and monitor patient data transmitted from the unit's monitoring and ventilation systems. In another form of the invention, monitoring system which fixed on the side rail may configured to communicate wirelessly (e.g., wireless antenna) and/or data connection (cable) to the wall-mounted monitor, also ventilation control box may be configured to communicate via wireless and/or data cable to the wall-mounted monitor.

The present disclosure overcomes the previous hazards and disadvantages, in one aspect, Intrahospital Transport comes more short time procedure because the staff doesn't need to disconnect the patient from a room equipped machines to portable equipment, they don't need to disconnect hoses and cables from the patient, the unit provided with all the systems which patient needs during (IHT). The staff only needs to disconnect the unit power cable and the other two connections in a few seconds, then the unit will be ready to transfer the patient, leading to safer (IHT). No more exposing patient life to danger by putting items behind him at bed surface, leading to all staffs attention will be only to the patient.

After the procedure finish, the staff brings the patient back to his ICU room, they only need to connect unit power cable and other two connections, unit automatically running on the main power supply and main gas supply, patient vital signs displays on the wall-mounted monitor.

Pendent system and other equipment around the bed won't be existed leading to provide more space around the patient for staff to move freely during all patient procedures. Other advantages, no more cables, wires, and hoses coming from outside the bed to patient annoying the staff and impeding their movements around the patient.

The present disclosure provides those advantages leading to saving efforts and time for staff, providing patients and staff with more safety procedures. Also expected that such a disclosure may save a part of ICU department expense, another advantage of this disclosure is that, building ICUs departments at temporary hospitals or emergency isolation departments during catastrophes or pandemic such as (covid19. e.g,) become easier, faster and inexpensive.

### BRIEF DESCRIPTION OF THE DRAWINGS

The detailed description particularly refers to the accompanying figures in which:

FIG. 1 illustrates an embodiment of a mobile integrated critical care patient apparatus with one or more systems connected to a patient.

FIG. 2A is a front view of the left head side rail.

FIG. 2B is a rear view of the left head side rail.

FIG. 3A is a front view of the left intermediate side rail.

FIG. 3B is a rear view of the left intermediate side rail.

FIG. 4A is a front view of the right intermediate side rail.

FIG. 4B is a rear view of the right intermediate side rail.

FIG. 5 is a front view of the right head side rail.

FIG. 6 is a simplified perspective view of an embodiment of the unit base frame and its components.

FIG. 7 illustrates a cross-section view of the head-end section with inspiration and expiration modules coupled to the patient.

FIG. 8 is a block diagram illustrating an embodiment of a patient breathing assistance system (ventilation system).

FIG. 9 is a block diagram illustrating the gases control box.

### DETAILED DESCRIPTION OF DRAWINGS

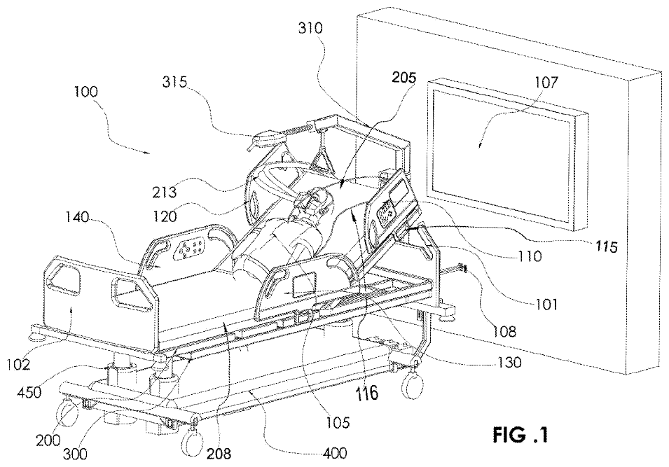

FIG. 1 illustrates an embodiment of a mobile integrated critical care apparatus 100, (also referred to as the unit 100), configurable to provide most of the patient needed systems inside the ICU room, connected to patient 105. In some embodiments, unit 100 includes a base frame 400, intermediate frame 300, and patient surface frame 200. A lifting system 450 coupled to the base frame 400 and supporting the intermediate frame 300, a retractable footboard 102, headboard 101, pair of head side rails assemblies left 110, right 120, and pair of intermediate side rails assemblies left 130, right 140. Each of the side rail assemblies 110, 120, 130, and 140 is individually movable between a raised position as shown in FIG. 1 and each movable to lower position (not shown).

according to this invention, the patient 105 is connected to different systems, such as a pneumatic system 455 (also referred to as ventilation system 455) delivers breathing gases to patient 105 via breathing circuit 213 (also referred to as patient limb 213). Another system is a patient monitoring system, which comprises a monitoring system interface 112 (also referred to as monitoring system display 112) configures to display patient vital signs data.

in other embodiments, the wall interface 107 (also referred to as wall-mounted monitor 107) may be configured to store and monitor all patient 105 data and systems performance which transmitted from the unit 100 monitoring and ventilation 455 systems. Wall-mounted monitor 107 may include memory, one or more processor, storage, and/or other components of the type commonly found in command computing devices. The monitor 107 may be implemented with hearing and sight types of alarm systems to provide alerts when necessary such as when patient or systems values out of setting values entered by the user. Also, the monitor 107 may be coupled to an arm that mounted to the wall. In other embodiment, a dual gas hose 481 coupled to main gases source 108 to supply the unit ventilation system 455 with needed gases (e.g., air and oxygen).

unit 100 includes a head motor or actuator coupled to backrest board 205 for moving the board 205 up or down to any needed degrees, more motors with different lengths coupled to other sections to control the movements of the patient surface frame 200 (not shown). Unit 100 is moved with the assist of internal motors controlled by means of interfaces and keypads 132, 114, 136, 143 that can be used by users like hospital staff or patient 105 to adjust the unit 100 in any needed position.

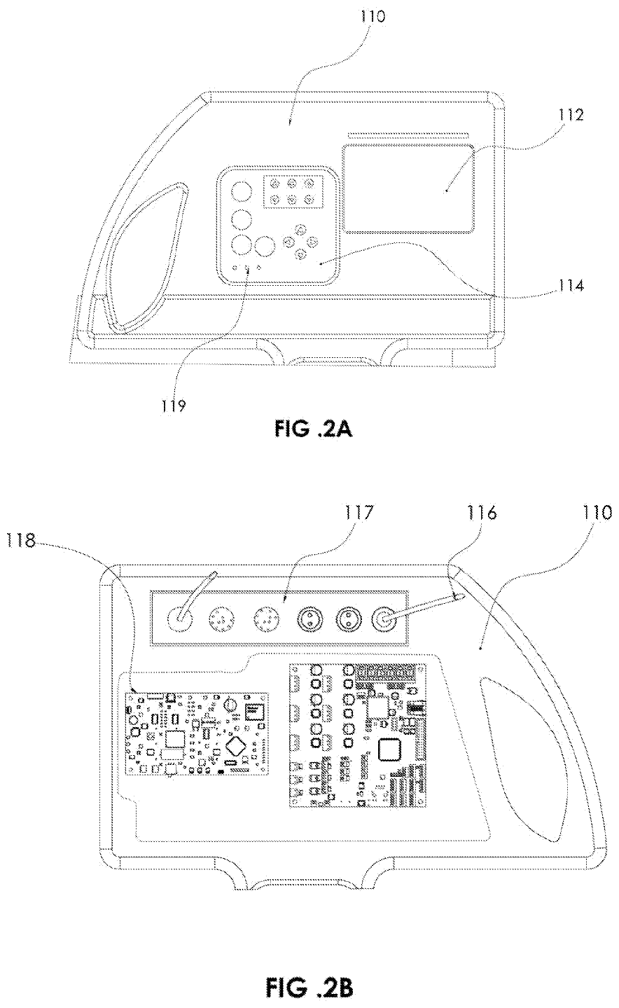

FIG. 2A illustrates a front view of the left head side rail 110 comprises two types of interfaces, unit control keypad 114 to enable the user from operating the unit 100 to any needed position. The keypad 114 includes indicators 119 a visual contact with the operator about the status of unit 100 such as unit power supply, battery conditions, and functions lock. Another interface, monitoring system display 112 which may be provided with touch-sensitive controls. The display 112 may be configured with a graphical, numerical, and/or textual elements to monitor patient 105 vital signs data.

FIG. 2B illustrates a rear view of the left head side rail 110 comprises a docking area 117, patient probes 116 and monitoring electronic system 118. Docking area 117 located at the top part of the side rail 110 and includes various types of outlets. The electronic system 118 located inside the side rail 110 may be connected to an air pump, valves, sensors, and/or other components needed for detecting patient vital signs and all located inside the side rail 110. The electronic system 118 configured to receive signals from patient 105 via probes 116 provided with sensors, the probes 116 couples to docking area 117 in order to communicate with the electronic system 118. The display 112 communicates with the system 118 and configured to monitor patient vital signs data. The system supplied with DC power supply from the additional power box 459 located at unit base frame 400.

when a critical care department received a patient 105 and required vital signs to be monitored, the patient 105 may be instrumented with a number of various types of probes 116, connecting the probes 116 to patient 105. For example, blood pressure cuff may be wrapped around patient 105 arm and other side of cuff connection connected to specific outlet at docking area 117 and same with finger cuff. Other example, the probes 116 may be placed at the appropriate positions on the patient 105 chest and abdomen, and the other side of the probes 116 connected at its outlet at docking area 117. (not shown)

the signals and data transmitted between the electronic system 118 and wall mounted 107 may be transferred via two ways: first, via data cable starting from the system 118 through side rail 110 lock mechanism 115 going through transit joint 470 to the unit base frame 400 structure till ended over the edge of head-end section 500 at data connector 503 position. Second, wirelessly (e.g., Wi-Fi antenna) connecting the system 118 with the monitor 107. (not shown)

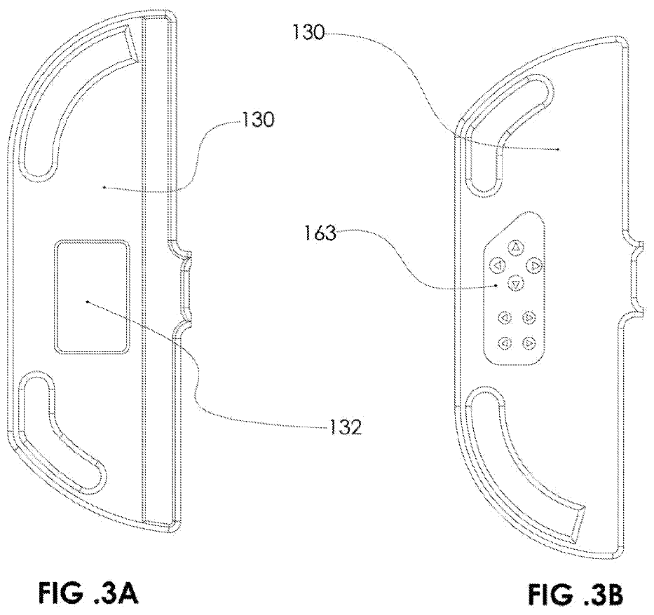

FIG. 3A illustrates left intermediate side rail 130 provide with bed control interface 132 (also referred to as unit control display 132), supported with touch-sensitive controls to enable the user from operating the unit 100 to any needed positions and movements. The display 132 communicates with bed circuitry and configured to display information of possible interest to caregivers concerning bed functions and features, for example, patient weight and/or patient safety alarms.

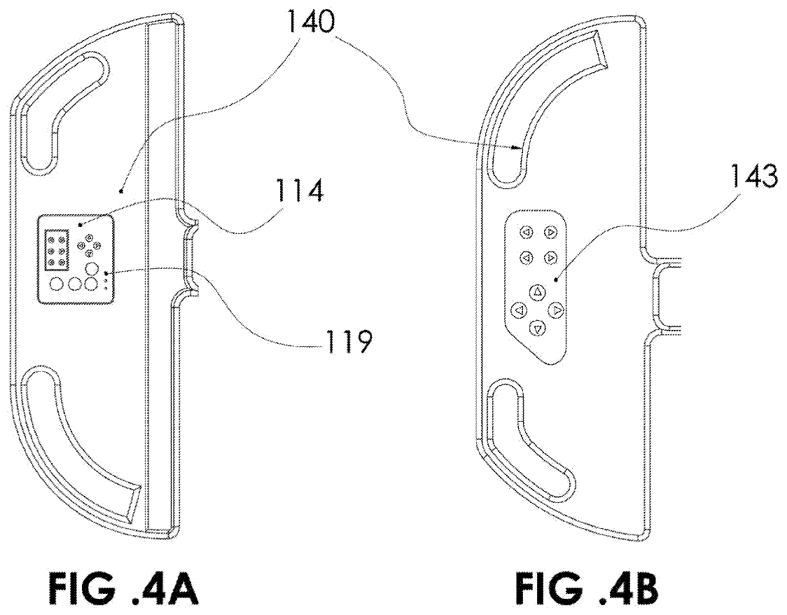

FIG. 3B illustrates a rear view of left intermediate side rail 130 includes a control keypad 136 with particular buttons for specific functions and movements which allow the patient 105 to use. Same details with FIG. 4B which illustrates the rear view of the right intermediate side rail 140 including the control keypad 143.

FIG. 4A illustrates a front view of the right intermediate side rail 140 includes unit control keypad 114 which provided with a various collection of buttons to control all the unit 100 movements. Same control keypad in FIG. 2A.

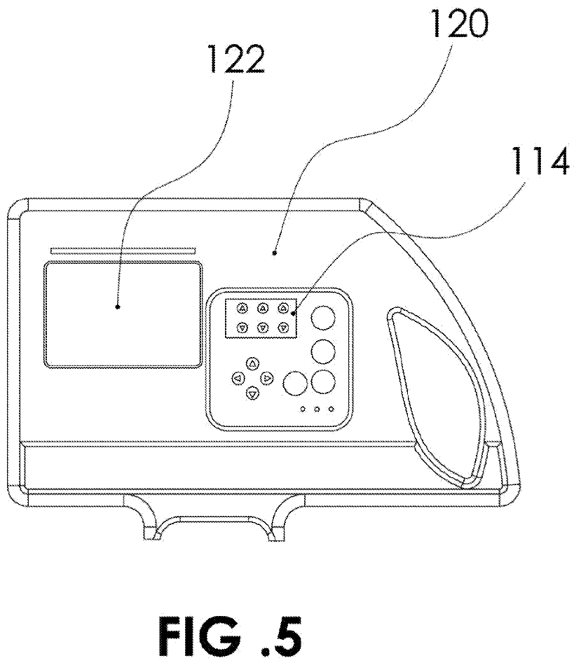

FIG. 5 illustrates the front view of the right head side rail 120 includes one of the ventilation system interfaces 122 (also referred to as ventilation system display 122). The display 122 may configure to monitor patient data, system performance in different forms, historical data, and capable of delivering commands to ventilation control box 460 based on user inputs. The display 122 may be provided with touch screen capability, allows the user to navigate through various display screens, make selections, modify and control various parameters. The display 122 may communicate to ventilation control box 460 via cables coming to the side rail 120 through surface and intermediate frames 200 and 300 then to transit joint 470 till base frame 400 that include the control box 460. The side rail 120 also includes a control keypad 114.

User interfaces 107, 112 and 122 are generally configured with a graphical, textual, numerical and touch screen elements as described, the display 112 at side rail 110 and the display 122 at side rail 120 are synchronized to provide the same data substantially at the same time to the wall-mounted monitor 107. The interfaces 107, 112, and 122 may be configured to show information about connectivity in both ways, by cable and/or wirelessly. Patient data may be tied or rendered together in a database record, may be resident in the same memory device, and/or may be linked together in fields of records in a database across a network.

Each side rail 110, 120, 130, and 140 couples to surface frame 200 through lock mechanism 115. All side rails have the same lock mechanism 115 design except the lock mechanism 115 for side rail 110 which drives the side rail 110 to full open from raised to a lowered position in two steps. The first step opens the side rail 110 near to half where the probes 116 which couples to docking area 117 still up to the patient mattress 208 level, after the user makes certain there are no probes 116 connected to docking area 117, then the user can go on with the second step to open the side rail 110 full to a lowered position. (not shown). This procedure to keep the probes 116 and connector at docking area 117 safe and not exposed to broken damages.

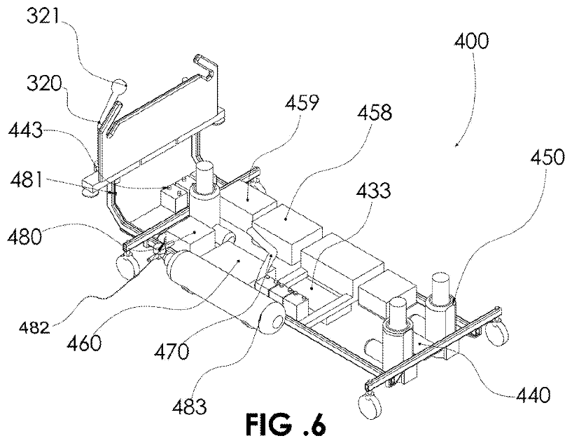

FIG. 6 illustrates a simplified perspective view of an embodiment of a base frame 400 which comprise a lifting system 450, gases control box 480, ventilation control box 460, ventilation power box 458, additional power box 459, drive wheel system 433, power storage device (e.g., rechargeable batteries) 443, holders for a backup interchangeable oxygen cylinder 483, bed power box 440, transit joint 470 and gases filters 482.

Adjusting the unit 100 high using a lifting system 450 which is actuators (e.g., electric motors) designs with column shape to provide extra space for other components at unit base frame 400. Lifting system 450 is substantially similar to the lifting system of (the Multicare® bed available from Linet.s.pol.company.inc), more details in U.S. Pat. No. 7,647,659 B2. Other aspects of the unit 100 structure may be similar to the Multicare® bed structure in more detail in U.S. Pat. Nos. 8,959,680 B2 and 8,112,836 B2, each of which is hereby expressly incorporated by reference herein.

In other embodiments, the ventilation control box 460 includes an electronic system to control the performance of unit ventilation system 455. The control box may include a processor, wireless module, memory, storage, and/or other components of the type commonly found in a command computing device. Control box 460 may deliver commands to gases control box 480 and (inspiratory and expiratory) modules 210 to control the ventilation provided to the patient 105, this specific commands may be based on inputs received from ventilation display 122 entered by user, sensors 487, patient 105 and/or other components of the system 455.

Ventilation power box 458 may include one or more converters (e.g., a DC/DC converter and/or an AC/DC converter), providing power supply to ventilation control box 460 and other ventilation system 455 components.

A rechargeable batteries 443 keeps the unit 100 and its systems remaining in operation without power interruption while the patient 105 being transported from location to other and/or when main power loss. In this embodiment, the ventilation display 122 is in communication with ventilation control box 460 and monitors the charge state, performance of batteries 443 and keeps batteries 443 in a charged state, same with unit's 100 other systems. (e.g., when patient transported the backup batteries 443 keeps all unit 100 systems (ventilation 455, monitoring, syringe, and infusion pumps, bed controls, drive wheel 433 and examination light 315) running for a long time enough for most of the transport procedures until patient 105 returned to his ICU room and unit 100 connected back to the main power supply).

Drive wheel system 433 couples to base frame 400 to assist the operator transporting the unit 100 easily with less effort. The system 433 may be similar to (I Drive Power® from Linet.s.pol.company.inc) described in more details in U.S. Pat. No. 10,206,835 B2.

In other embodiments, additional power box 459 may include one or more converters (e.g., a DC/DC converter and/or an AC/DC converter), providing power supply to monitoring system which located inside the side rail 110, injection system (syringe and infusion) pumps and examination light 315. Same with bed power box 440 supplying bed control circuitry (not shown) with power.

In other embodiment, cables and gases hoses passage between components at base frame 400 and components at intermediate and surface frames 300 and 400 through a flexible transit joint 470.

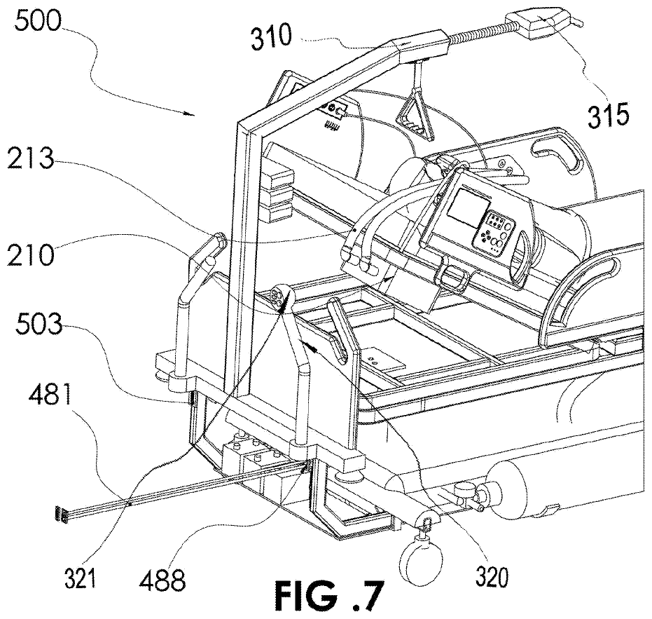

FIG. 7 illustrates across-section of unit 100 includes head-end section 500 and patient breathing circuit 213 carrying gas mixture to and from patient 105 and couples to (inspiration and expiration) modules 210. The circuit 213 may include a (single-lamb or dual-lamb), in the present embodiment, using a dual-lamb 213, both inspiratory and expiratory lambs 213 couples the patient 105 to ventilation system 455.

In other embodiment, inspiration and expiration modules 210 located at the rear side of patient backrest board 205 to provide the ventilation system 455 with more flexibility during all unit 100 positions and movements. Modules 210 and patient circuit 213 may include one or more sensors 487 for detecting one or more parameters related to the ventilation of the patient 105. Sensors 487 may include one or more devices for detecting different parameters of gas flowing from or to patient 105, (e.g., gases pressure, flow rates, flow volume, the humidity of gases, and/or temperature).

In other embodiment, head-end section 500 may include, a gases inlet connector 488 couples to the edge of head-end section 500 connecting the main gases source 108 to unit ventilation systems 455 through dual gas hose 481. At the same edge located a data connection 503 for connecting monitoring electronic system 118 and ventilation control box 460 to wall-mounted monitor 107.

Head-end section 500 may include a drive wheel controller 321 couples to push hands 320, configures to enable the unit 100 to be transported with drive wheel system 433 and/or manually.

In other embodiment, the head-end section 500 may include a removable bar 310 implemented with DC power supply and bracket holder for syringe and infusion pumps. The bar 310 supported with a flexible arm ended with patient examination light 315. The bar 310 located at head-end section 500 near to the left side (not at the center of a head-end section 500) to provide the user with a clear view during transporting the unit 100 with any barriers in front of his sight.

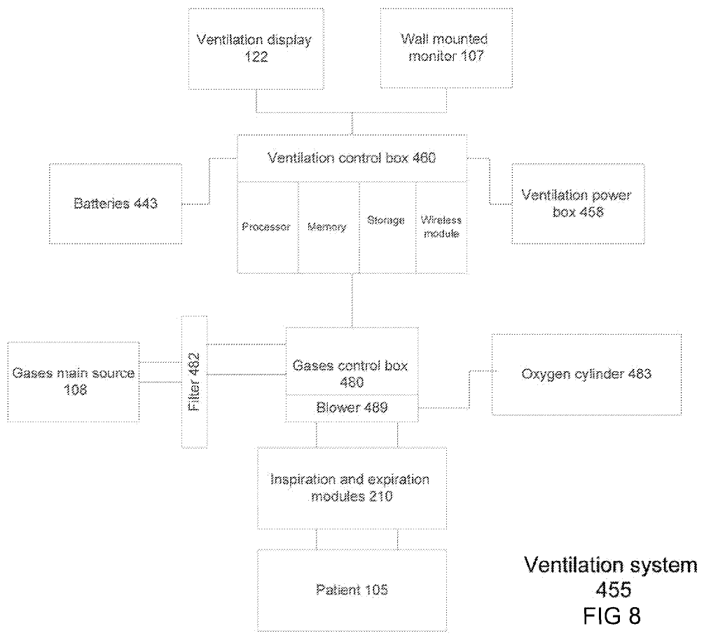

FIG. 8 is a diagram illustrating the components and stages of ventilation system 455 which provides breathing support to patient 105 who might unable to breathe sufficiently. The system 455 comprises a ventilation control box 460, display 122, batteries 443, ventilation power box 458, gases control box 480, blower 489, oxygen cylinder 483, inspiration and expiration modules 210 and patient breathing circuit 213.

In the present embodiment, the system 455 starts with dual gas hose 481, from one side couples to main gases source 108 and the other side coupled to gas inlet connector 488 located at head-end section 500. The dual hose 481 passes through filters (may include water trap) 482 before entering the gases control box 480 as shown in FIG. 6. Gases control box 480 supported with air backup source (blower 489) delivering air to the system 455 during patient transport and/or any emergency event could happen to main source 108. Also, the system 455 supported with an interchangeable oxygen cylinder 483 located at the right side of base frame 400 supporting the system 455 during patient transport and/or any emergency event could happen to main source 108. So blower 489 and oxygen cylinder 483 are backup sources of pressurized gases controlled by gases control box 480.

The control box 460 is in communication with ventilation display 122 and wall-mounted monitor 107 to display patient parameters, system performance, and alarms. Control box 460 may also control the operation of back up batteries 443 provided for the system 455. For example, in an embodiment, the control box 460 may control the charging of batteries 443 when they are not in use, in yet another embodiment, the ventilation display 122 may include monitoring and issuing alarms related to batteries 443 status.

The ventilation display 122 is a touch-sensitive, able to serve both as an input user interface and an output device. The display 122 is configured to display any type of ventilation information such as sensors 487 reading, parameters, commands, alarms, warnings, etc. the display 122 enable the operator to interact with the system 455 (e.g., changing ventilation setting, select operational modes, view monitored parameters, etc.)

Inspiration and expiration modules 210 may deliver the needed volume of gas mixture to the patient 105 via breathing circuit 213. Modules 210 couples to gases control box 480, blower 489, and oxygen cylinder 483 via dual hose 481 to control the pressurized breathing gases. Also modules 210 couples to ventilation control box 460 in order to control system performance by virtue of data receiving from sensors 487.

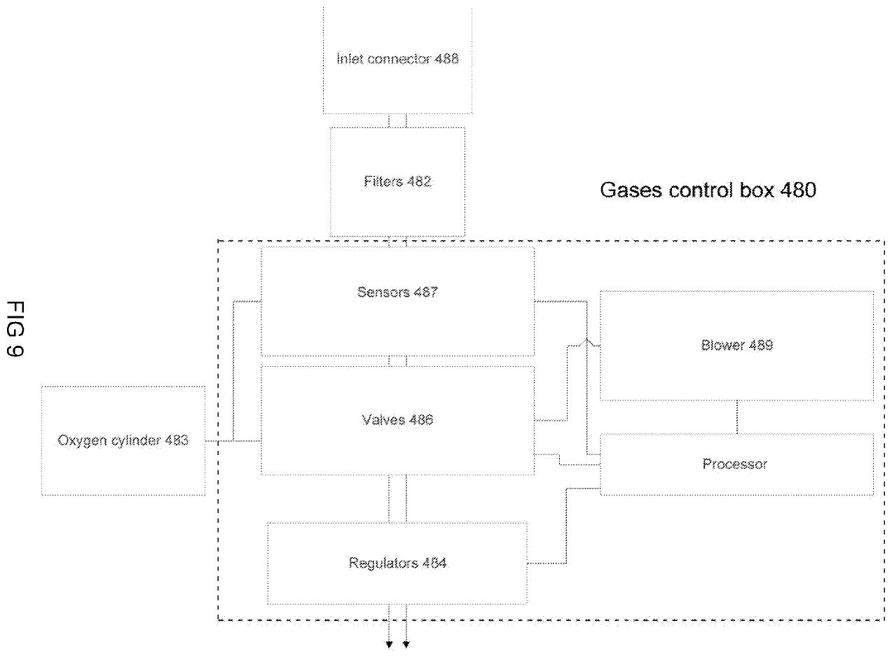

FIG. 9 illustrates the components of gases control box 480. In the present embodiment, pressurized air and oxygen sources 108 available from wall outlets. Delivering gases to unit ventilation systems 455 going through dual gas hose 481, dual hose 481 couples to unit 100 via gas inlet connector 488, hose 481 pass by head-end structure 500 till entered filters 482 (different types such as water trap) which couples to the structure of base frame 400 then, gases delivered to gases control box 480.

Gases control box 480 makes the system 455 capable of operating independently of external sources of pressurized air and oxygen, the box 480 may include a variety of other components such as valves 486, regulators 484, sensors 487, source of pressurized air (blower 489), a connection to oxygen cylinder 483, filters, tubing, etc.

The ventilation control box 460 is in communication with gases control box 480 and configures to control the operation and functions of gases control box 480. The ventilation control box 460 configures to operate the valves 486 inside box 480 to control switching between gases from main source 108 and backup sources 489, 485, this control effected by pressure and flow rate signals which received from sensors 487. In other embodiment, pressure regulators 484 to regulate the flow of gases in a desired pressure and rates before delivering the gases to inspiration and expiration modules 210.

Sensors 487 may be located at one or more various locations at the ventilation system 455 for monitoring the pressure and flow of gases flowing into and/or out from patient 105 and ventilation system 455. For example, one or more sensors 487 may be located in gases control box 480, inspiration and expiration modules 210 and/or patient circuit 213. Ventilation control box 460 may be operable to control the operation of ventilation system 455 based on various inputs received from the operator via ventilation display 122 and/or data received from one or more sensors 487. For example, ventilation control box 460 may regulate the pressure and flow of gas delivered to patient 105 based on data (signals) received from sensors 487.

It will be clear that the unit and systems described herein are well adapted to attain the ends and advantages mentioned as well as those inherent therein. While various embodiments have been described for purposes of this disclosure, it's not intended to be limited to the details shown, various structural changes and modifications may be made which are well within the scope of the present invention, numerous other changes May be made, which will readily suggest themselves to those skilled in the art and which are encompassed in the spirit of the invention and as defined in the appended claims.

1.-9. (canceled)

10. A mobile integrated patient support apparatus used as a mobile integrated critical care unit for patient sleep and transport with multiple uses inside and outside the hospital, comprising:
a patient monitoring system;
a patient ventilation system;
a removable bar used as a holder for syringe and infusion pumps and provided with outlets of power supply for syringe and infusion pumps, the removable bar is further ending with a fixable arm supported with an examination light;
four side rails couple to the patient surface frame; and
a base frame containing several components.

11. The apparatus of claim 10, wherein the patient monitoring system comprises patient probes connected to the patient from one side, while the other side is connected to the monitoring electronic system via outlets of a docking area, wherein the monitoring electronic system is configured to receive and process data from the patient probes and monitor it through a monitoring system display.

12. The apparatus of claim 11, wherein the monitoring electronic system further comprises a monitoring system display located at outer side of a left head side rail and is configured to display the patient's vital signs.

13. The apparatus of claim 11, wherein the monitoring electronic system is located inside a left head side rail.

14. The apparatus of claim 11, wherein the docking area is located at the rear of a left head side rail.

15. The apparatus of claim 10, wherein the patient ventilation system comprising:
a gases inlet connector;
a gases control box;
a ventilation control box and a power control box; and
inspiration and expiration modules;
a ventilation system display.

16. The apparatus of claim 15, wherein the gases inlet connector is coupled to an edge of the base frame from a side of a head-end section connecting the gases control box to a gases main source coupled to the wall.

17. The apparatus of claim 15, wherein the gases control box is located at the base frame and includes several components for controlling the supplying gases for the ventilation system and for switching between the main sources coupled to the wall and a backup source comprising an air blower and a replaceable oxygen cylinder.

18. The apparatus of claim 15, wherein the patient ventilation system further comprises separated ventilation power and control boxes attached to the base frame, wherein the control box is in communication with all the patient ventilation system components controlling the operation of the system, and a flexible transit joint passing gases hoses and cables between components at the base frame to inspiration and expiration components which are coupled to the patient surface frame.

19. The apparatus of claim 15, wherein the inspiration and expiration modules are integrated as part of the patient ventilation system located at the rear of a patient backrest board and supported with a breathing circuit coupled to the patient.

20. The apparatus of claim 15, wherein the patient ventilation system comprises a ventilation system display located at an outer side of right head side rail and configured to display ventilation system data and in communication with the ventilation control box.

21. The apparatus of claim 10, wherein the base frame supported with data connection is coupled to an edge of a head-end section configured to communicate between a ventilation control box and the monitoring electronic system with a wall-mounted monitor.

22. The apparatus of claim 10, wherein the patient surface frame comprising a left head side rail is configured to moves between a raised position and a lowered position in two stages, first opening the side rail to halfway position in which patient probes are connected to a docking area up to the level of the patient mattress, and then in a second step in which the user ensures that the patient probes are not connected to sockets of the docking area, opening said rail fully to the lowered position through a different mechanism.
